(19) United States
(12) Patent Application Publication
(10) Pub. No.: US 2009/0029984 A1
(43) Pub. Date: Jan. 29, 2009

(54) SYNERGISTIC COMBINATION FOR THE TREATMENT OF PAIN (CANNABINOID RECEPTOR AGONIST AND OPIOID RECEPTOR AGONIST)

(75) Inventors: Julia Adam-Worrall, Central Scotland (GB); David Robert Hill, Central Scotland (GB); Jean Cottney, Central Scotland (GB)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon, Oss (NL)

(21) Appl. No.: 11/995,243

(22) PCT Filed: Jul. 6, 2006
(86) PCT No.: PCT/EP06/63985

§ 371 Date: Jan. 10, 2008

(30) Foreign Application Priority Data
Jul. 11, 2005 (EP) 05106309.7

(51) Int. Cl. A61K 31/485; A61K 31/5377; A61K 31/445; A61K 31/135; A61P 29/00

(52) U.S. Cl. 514/232.8; 514/283; 514/326; 514/330; 514/648; 514/659

(57) ABSTRACT

The invention relates to a pharmaceutical dosage form comprising an analgesic combination for simultaneous or sequential use which comprises a peripherally restricted cannabinoid CB1 receptor agonist having a brain Cₘₐₓ to plasma Cₘₐₓ ratio of less than 0.1 and an opioid receptor agonist, as well as to a method for treating pain using said pharmaceutical dosage form.

The present invention relates to the field of analgesic combinations, more specifically to the synergistic combination of a peripherally restricted cannabinoid receptor agonist with an opioid receptor agonist and to the use of this combination in the treatment of pain.

Pain treatment is often limited by the side effects of currently available medication. For moderate to severe pain, opioid receptor agonists (opioids) are widely used. The best known compounds in this group are morphine, codeine, pethidine, tramadol, sufentanil and fentanyl. These agents are cheap and effective but suffer from serious side effects, which comprise dependence (both physical and psychological), respiratory depression, muscle rigidity, disorientation, sedation, nausea, vomiting, constipation, pruritis and urinary retention. These distressing side effects limit the doses of opioids that can be used, frequently resulting in patients receiving sub-optimal pain control.

Owing to the severity of side effects, combinations of opioid receptor agonists with other analgesic drugs have been studied as a method to decrease the dose of the opioid. Analgesic drugs that have been considered in this respect are the non-steroidal anti-inflammatory drugs (NSAIDs), such as aspirin, ketorolac and ibuprofen, COX-2 selective inhibitors such as meloxicam and celecoxib, and paracetamol. It has been reported in the scientific literature that a decrease in the dose of opioid analgesics is possible with concurrent administration of NSAIDs. Cataldo P. A. et al. (Surg. Gynecol. Obstet. 176: 435-438, 1993), Picard P. et al. (Pain 73: 401-406, 1997), See W. A. et al., (J. Urol. 154: 1429-1432, 1995) and several others report the effects of a combination of morphine and ketorolac trometamol (Toradol) in post-operative pain relief. This combination was also found to be effective for pain treatment in cancer patients (Joishy S. K. and Walsh D., J. Pain Symptom Manag. 16: 334-339, 1998). Gupta A. et al (Reg. Anesth. Pain Med. 24: 225-230, 1999) suggest that this combination could be said to have a synergistic analgesic effect, while Sevarino F. B. et al. conclude that this combination has an additive effect. There is some evidence in the literature to suggest that intravenous propacetamol (a pro-drug of paracetamol) has a morphine sparing effect in post-operative pain (Binhas M. et al., BMC Anesthesiology 4: 6, 2004; Aubrun F. et al., Br J. Anaesth. 90: 314-319, 2003), although no reduction in morphine-related adverse effects was seen. Other published studies did not show significant differences in morphine consumption between intravenous propacetamol and placebo groups (Varassi G. et al., Anesth. Analg. 88: 611-616, 1999; Fletcher D. et al., Can. J. Anaesth. 44: 479-485, 1997; Siddik S. M. et al., Reg. Anesth. Pain Med. 26: 310-315, 2001).

NSAIDs and COX-2 inhibitors have limited efficacy in the treatment of moderate to severe pain and are not active in pre-clinical threshold models of antinociception, such as the tail flick test in rodents. Strong analgesics, such as centrally acting opioids, show robust activity in the tail flick test. There is preclinical evidence for synergy in the tail flick test between some combinations of opioids and NSAIDs, but not others. For example, ibuprofen has been shown to enhance the effects of the opioid agonists hydrocodone and oxycodone in the mouse tail flick test, whereas neither aspirin nor ketorolac influenced hydrocodone actions and ibuprofen did not potentiate fentanyl or morphine analgesia (Zelcer, S. et al., Brain Res. 1040: 151-156, 2005). The NSAID indomethacin and the selective COX-2 inhibitor NS-398, which were inactive by themselves in the rat tail flick test, did not enhance the antinociceptive effect of morphine in this model (Wong C—S et al., Br. J. Anaesthesia 85: 747-751, 2000).

Evidence is accumulating that cannabinoid receptor agonists have potential as analgesic and anti-inflammatory agents. Two types of cannabinoid receptors are implicated, the cannabinoid CB1 receptor, which is located primarily in the central nervous system (CNS) but which is also expressed by peripheral neurones and other peripheral tissues, and the cannabinoid CB2 receptor, which is mostly located in immune cells (Howlett, A. C. et al., International Union of Pharmacology. XXVII. Classification of Cannabinoid Receptors. Pharmacol. Rev. 54: 161-202, 2002). It has been suggested that peripherally restricted cannabinoid receptor agonists may be useful in the treatment of pain, without the side-effects associated with activation of CB1 receptors in the CNS, such as sedation and psychotropic effects (Piomelli D. et al., Nature 394: 277-281, 1998; Ko M-C and Woods J. H., Psychopharmacology 143: 322-326, 1999; Fox A. et al., Pain 92: 91-100, 2001; Johanek L. M. and Simone D. A., Pain 109: 432-442, 2004; Fox A. and Bevan S., Expert Opin. Investig. Drugs 14: 695-703, 2005). In contrast to compounds that activate CB1 receptors in the CNS, however, peripherally restricted cannabinoid receptor agonists, administered at doses that do not result in sufficient brain levels to activate CB1 receptors in the CNS, are not active in threshold models of antinociception such as the tail flick test. Therefore, these agents may not have sufficient efficacy to treat moderate to severe pain when administered alone. It is documented in the literature that centrally acting cannabinoid receptor agonists interact with opioid receptor agonists in a synergistic manner through interactions at the spinal and supraspinal level (Tham S. M. et al., Br. J. Pharmacol. 144: 875-884, 2005; Cichewicz D. L., Life Sciences 74:1317-1324, 2004; Pertwee R. G., Prog. Neurobiol. 63: 569-611, 2001; Welch et al., J. Pharmacol. Exp. Ther. 272: 310-321, 1995). The centrally acting cannabinoid receptor agonists which have been studied in combination with opioid receptor agonists (see J. D. Richardson, J. of Pain Vol 1, No. 1, 2-14, 2000) are characterized by a high brain Cₘₐₓ to plasma Cₘₐₓ ratio on systemic or local application. The brain-to-plasma ratio in the rat for the cannabinoid agonists Δ⁹-tetrahydrocannabinol (Δ⁹THC), cannabinol and cannabidiol were reported to be 0.96, 0.88 and 2.61, respectively (Alozie, S. O. et al, Pharmacology, Biochem. Behav. 12: 217-221, 1980), while Dyson et al (Pain, 116: 129-137, 2005) reported a brain-to-plasma ratio in rat of 1.0 for Δ⁹THC and of 1.3-1.9 for the aminoalkylindole cannabinoid agonist WIN55, 212-2.

Synergistic effects would not be expected for a combination of an opioid receptor agonist with a peripherally restricted cannabinoid receptor agonist having a very low capacity to penetrate into the brain and spinal cord.

There remains an unmet medical need for an analgesic agent, or combination of agents, that produces effective analgesia in moderate to severe pain with reduced side effects compared to currently available therapies.

It is an object of the present invention to provide a pharmaceutical dosage form which comprises a peripherally restricted cannabinoid CB1 receptor agonist having a brain Cₘₐₓ to plasma Cₘₐₓ ratio of less than 0.1, as measured in mice on intravenous administration, and an opioid receptor agonist for simultaneous or sequential use. In the pharmaceutical dosage form of the invention the peripherally restricted cannabinoid CB1 receptor agonist can enhance the antinociceptive effect of the opioid receptor agonist in a synergistic manner. Such dosage forms allow for a reduced dose of the opioid receptor agonist to be administered, thereby reducing its plasma concentration while still providing effective pain treatment. This provides an opportunity to reduce the side effects and the dependence and tolerance which the patient may experience when subjected to acute or prolonged treatment with opioid receptor agonists.

In reference to the present invention, the term “cannabinoid receptor” is intended to encompass CB1 and CB2 receptors. The term “cannabinoid receptor agonist” is intended to encompass CB1 and CB2 receptor agonists, including compounds that are essentially non-selective for CB1 versus CB2 and compounds that show varying degrees of selectivity for either the CB1 receptor or the CB2 receptor. In a preferred embodiment of the invention, the cannabinoid receptor agonists of the invention are CB1 receptor agonists.

The term “peripherally restricted cannabinoid CB1 receptor agonist” encompasses cannabinoid CB1 receptor agonists that, when given by the intended route of administration at the intended dose, activate cannabinoid CB1 receptors in peripheral neurones and other peripheral tissues, but do not significantly activate cannabinoid CB1 receptors in the CNS. A peripherally restricted cannabinoid receptor agonist has a sufficiently low penetration of the blood-brain barrier when administered by the intended route at the intended dose that the maximum concentration of the compound in the CNS is lower than that required for significant activation of central CB1 receptors.

A peripherally restricted cannabinoid CB1 receptor agonist according to the invention is characterized and can be identified from a ratio of maximum concentration in the brain to maximum concentration in plasma which is less than 0.1, as measured in a mouse after intravenous dosing. The preferred peripherally restricted cannabinoid CB1 receptor agonists have a brain Cₘₐₓ to plasma Cₘₐₓ ratio which is less than 0.05. Especially preferred peripherally restricted cannabinoid receptor agonists have a brain Cₘₐₓ to plasma Cₘₐₓ ratio which is less than 0.025.

In-silico models to predict the ability of compounds to cross the blood-brain barrier have been described in the literature (Clark D. E., DDT(2003) δ: 927-933). These models may be used to determine whether a particular cannabinoid receptor agonist is likely to be peripherally restricted, based on its chemical structure. For example, an inverse relationship between polar surface area (PSA) and brain penetration has been described, such that compounds with a PSA greater than 70 Å² are considered likely to have low brain penetration (Kelder J. et al., Pharm. Res., 16: 1514-1519, 1999). The PSA is a measure of a molecule's hydrogen bonding capacity and is commonly calculated by summing the contributions to the molecular surface area from oxygen and nitrogen atoms and hydrogens attached to oxygen and nitrogen atoms.

With reference to the present invention, the term “opioid receptor agonist” is intended to encompass all drugs with morphine-like actions. The opioids are a group of drugs, both natural and synthetic, that are employed primarily as centrally-acting analgesics and are opium or morphine-like in their properties. The opioids include morphine and morphine-like homologs, including e.g. the semisynthetic derivatives codeine (methylmorphine) and hydrocodone (dihydrocodeinone) among many other such derivatives. Morphine and related opioids exhibit agonist activity at p-opioid receptors as well as 5 and K opioid receptors, to produce analgesia. In addition to potent analgesic effects, the opioid receptor agonists may also cause a number of undesirable effects, including, for example, respiratory depression, nausea, vomiting, dizziness, drowsiness, mental clouding, dysphoria, pruritis, constipation, increased biliary tract pressure, urinary retention and hypotension.

Examples of opioid receptor agonists suitable for the present invention include alfentanil, allylprodine, alphaprodine, anileridine, benzylmorphine, bezitramide, buprenorphine, butorphanol, donitazene, codeine, cyclorphan, desomorphine, dextromoramide, dezocine, diampromide, dihydrocodeine, dihydromorphine, eptazocine, ethylmorphine, fentanyl, hydrocodone, hydromorphone, hydroxypethidine, levophenacylmorphan, levorphanol, lofentanil, methadone, meperidine, methylmorphine, morphine, nalbuphine, necomorphine, normethadone, normorphine, opium, oxycodone, oxymorphone, pentazocine, pholcodine, profadol, sufentanil and tramadol.

Preferred opioid receptor agonists for use in the invention are morphine, codeine, fentanyl, oxymorphine, oxycodone, hydromorhine, methadone and tramadol. Especially preferred opioid receptor agonists for use in a pharmaceutical dosage form of the invention are morphine, codeine, fentanyl and tramadol.

The peripherally restricted cannabinoid CB1 receptor agonist and the opioid receptor agonist can be administered subsequentially (in any order) or at the same time. It is also possible to administer both drugs in various administration forms, i.e. either or both may be administered by intravenous bolus or infusion, subcutaneously, intramuscularly, orally, rectally or sublingually. In a preferred mode the present invention provides for pharmaceutical dosage forms for oral administration. Dosage levels of the cannabinoid receptor agonist from about 0.005 mg to about 100 mg per kilogram of body weight per day may be therapeutically effective in combination with an opioid analgesic.

The combination of the pharmaceutical dosage forms of the invention comprises as the active ingredients the peripherally restricted cannabinoid CB1 receptor agonist and the opioid receptor agonist either in separate dosage forms for each agonist or in a dosage form comprising both of the agonists.

For oral administration, the active ingredients of the analgesic combination of the invention may be presented as discrete units, such as tablets, capsules, powders, granulates, solutions, suspensions, and the like.

For, parenteral administration, the active ingredients of the pharmaceutical dosage form of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

Mixed with such pharmaceutically acceptable auxiliaries, e.g. as described in the standard reference, Gennaro, A. R. et al., Remington: The Science and Practice of Pharmacy (20th Edition., Lippincott Williams & Wilkins, 2000, see especially Part 5: Pharmaceutical Manufacturing), the active ingredients may be compressed into solid dosage units, such as pills, tablets, or be processed into capsules or suppositories. By means of pharmaceutically acceptable liquids the active ingredients can be applied as a fluid composition, e.g. as an injection preparation, in the form of a solution, suspension, emulsion, or as a spray, e.g. a nasal spray.

For making solid dosage units, the use of conventional additives such as fillers, colorants, polymeric binders and the like is contemplated. In general any pharmaceutically acceptable additive which does not interfere with the function of the active ingredients can be used. Suitable carriers with which the active ingredients of the invention can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes an analgesic combination, as hereinbefore described, in combination with packaging material suitable for said combination, said packaging material including instructions for the use of the combination for the use as hereinbefore described.

The pharmaceutical dosage forms of the invention are suitable for the treatment of pain. Any analgesic treatment is indicated, but the compositions of the invention are particularly useful in the treatment or prophylaxis of moderate to severe pain for which opioid drugs would normally be indicated, such as treatment of peri-operative pain, pain in neoplastic patients, pain in terminal patients, chronic pain (including back pain, neuropathic pain and inflammatory pain such as arthritis), obstetric pain and dysmenorrhea.

### EXPERIMENTAL

### Preparation of Peripherally Restricted CB1 Receptor Agonists

2-(2-Hydroxy-ethylcarbamoyloxymethyl)-5,7-dimethyl-3-(2-methylsulfamoylphenyl)-4-oxo-3,4-dihydro-quinazoline-6-carboxylic acid ethyl ester, compound 1a, was prepared as described in the International Patent Application WO2003066603 (Novartis Pharma GMBH).

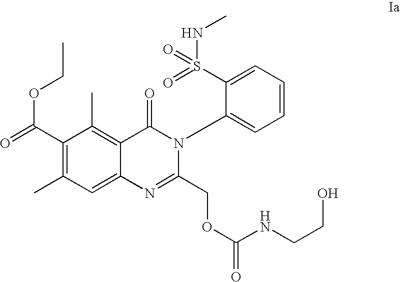

(S)-7-Chloro-3-[(5-{[3-N-(2-hydroxyethyl)carboxamido]piperidin-1-yl}methyl)([1,2,4]-thiadiazol-3-yl)]-1-(1,1-dioxo-hexahydrothiopyran-4-yl)methyl-1H-indole, hydrochloride salt, compound 2, was prepared as described below:

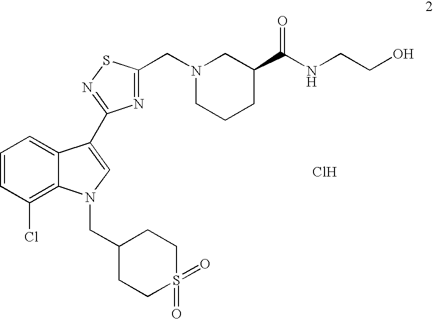

### Step A: Tetrahydrothiopyran-4-carbonitrile

A mixture of tetrahydrothiopyran-4-one (75 g, 646 mmol) and toluenesulfonyl-methyl isocyanide (138.6 g, 710 mmol) in dimethoxyethane (2.5 L) was cooled to 0° C. and a solution of potassium tert-butoxide (145 g, 1.29 mol) in tert-butanol (1.3 L) added dropwise. The mixture was then allowed to warm to room temperature and stirred for 3 h before dilution with diethylether (3 L), washing with saturated sodium bicarbonate (2×1.5 L) and drying over magnesium sulfate. Removal of the solvent in-vacuo gave tetrahydrothiopyran-4-carbonitrile as a pale brown oil (88.3 g, 646 mmol).

### Step B: Tetrahydrothiopyran-4-carboxylic acid

A solution of tetrahydrothiopyran-4-carbonitrile (646 mmol), in ethanol (600 ml) was added in one portion to a rapidly stirring mixture of sodium hydroxide (258.4 g, 6.46 mol) in water (1.1 L). The mixture was then heated to 90° C. for 2 h, cooled to 0° C. and the pH adjusted to 2 with conc. hydrochloric acid solution. The ethanol was then removed in-vacuo and the suspension extracted into dichloromethane (3×1 L). The combined organic extracts were then dried over magnesium sulfate and evaporated in-vacuo to give tetrahydrothiopyran-4-carboxylic acid as a brown solid (96 g, 646 mmol).

### Step C: (Tetrahydrothiopyran-4-yl)methanol

A solution of borane dimethylsulfide complex (73.5 ml, 775 mmol) in anhydrous tetrahydrofuran (1.5 L) was treated dropwise over 15 min with a solution of tetrahydrothiopyran-4-carboxylic acid (646 mmol) in anhydrous tetrahydrofuran (300 ml). The mixture was then heated to 70° C. for 2 h, cooled to room temperature and quenched by dropwise addition of water until effervescence ceased. A further portion of water (500 ml) was then added and the tetrahydrofuran removed in-vacuo. The residue was then acidified with dilute hydrochloric acid solution and extracted into dichloromethane (3×500 ml). The combined organic layers were then dried over sodium sulfate and the solvent removed in-vacuo to give (tetrahydrothiopyran-4-yl)-methanol as a brown oil (90.18 g, 646 mmol).

### Step D: (1,1-Dioxo-hexahydro-1-thiopyran-4-yl)-methanol

A solution of sodium periodate (304 g, 1.42 mol) in water (3 L) was treated with a solution of (tetrahydrothiopyran-4-yl)-methanol (646 mmol) in methanol (1.7 L) and the mixture heated to 60° C. for 3 h. Sodium periodate (10 g) was then added and heating continued for a further 1 h before removal of all volatiles in-vacuo. The resulting granular residue was then shaken with succesive portions of diethyl ether (2×500 ml), dichloromethane (2×500 ml) and 50% (v/v) dichloromethane in methanol (2×500 ml). The remaining residue was then subjected to a continous extraction using dichloromethane for 18 h and the solvent combined with the earlier solvent extractions, dried over sodium sulfate and evaporated in-vacuo to give (1,1-dioxo-hexahydro-1-thiopyran-4-yl)methanol as an orange oil (106.2 g, 646 mmol) which crystallised on standing.

### Step E: Toluene-4-sulfonic acid 1,1-dioxo-hexahydro-1-thiopyran-4-ylmethyl ester

A solution of (1,1-dioxo-hexahydro-1-thiopyran-4-yl)-methanol (105 g, 640 mmol), pyridine (155 ml, 1.92 mol) and 4-dimethylaminopyridine (2.5 g, 20.5 mmol) in chloroform (1.5 L) was treated portionwise with p-toluenesulfonyl chloride (244 g, 1.28 mol) over 15 mins. The mixture was the stirred for 72 h, washed with water (2×1 L), saturated sodium chloride solution (1 L) and dried over sodium sulfate. The organic solvent was removed in-vacuo and the oily residue shaken with 60% (v/v) heptane in ethyl acetate to give a brown solid on filtration. This was dissolved in the minimum dichloromethane, passed through a celite pad eluting with ethyl acetate (4 L). The solvent was then removed in-vacuo until the solution volume was 750 ml and heptane (1.5 L) added. The resulting suspension was then filtered to give the title compound as a sandy coloured solid (130 g, 408 mmol).

### Step F: 7-Chloro-1-[(1,1-dioxohexahydrothiopyran-4-yl)methyl]-1/H-indole

A solution of 7-chloroindole (45 g, 296 mmol) in dimethylformamide (450 ml) was treated portionwise with sodium hydride (60% dispersion in mineral oil; 17.8 g, 444 mmol). The mixture was stirred at room temperature for 30 minutes. Toluene-4-sulfonic acid 1,1-dioxo-hexahydro-1-thiopyran-4-ylmethyl ester (95.45 g, 300 mmol) was then added portionwise over 15 minutes and the mixture stirred at room temperature for 72 h. The reaction was quenched with water (2 L) and the precipitate filtered off, washing with water (3×300 ml) and dried to afford the title compound as a colourless solid (79 g, 266 mmol).

### Step G: 7-Chloro-1-[(1.1-dioxo-hexahydrothiopyran-4-yl)methyl]-1H-indole-3-carboxylic acid

A solution of 1-[(1,1-dioxohexahydrothiopyran-4-yl)methyl]-7-chloro-1H-indole (79 g, 266 mmol) in dimethylformamide (800 ml) was cooled in an acetone/ice bath under nitrogen and trifluoroacetic anhydride (74.3 ml, 532 mmol) was added dropwise, maintaining the temperature below 5° C. The mixture was allowed to warm to room temperature with stirring over 2 h, and then quenched with water (3 L). The resulting 7-chloro-1-[(1,1-dioxo-hexahydrothiopyran-4-yl)methyl]-3-[(trifluoromethyl)-carbonyl]-1H-indole precipitate was filtered off, washing with water (3×700 ml). The damp solid was suspended in ethanol (500 ml), 4 M aqueous sodium hydroxide (500 ml) was added and the mixture was heated to reflux with stirring for 2 h. The mixture was cooled and the ethanol removed in-vacuo. Water (500 ml) and heptane (200 ml) were added and the mixture acidified to pH 2 with 5M aqueous hydrochloric acid. The suspension was filtered off, washing with water (3×500 ml) and dried to afford the title compound as a light brown solid (70 g, 205 mmol).

### Step H: 7-Chloro-1-[(1,1-dioxo-hexahydrothiopyran-4-yl)methyl]-1H-indole-3-carboxamide

A solution of 7-chloro-1-[(1,1-dioxo-hexahydrothiopyran-4-yl)methyl]-1H-indole-3-carboxylic acid (70 g, 205 mmol) in tetrahydrofuran (750 ml) was cooled to 0° C. under nitrogen and oxalyl chloride (23 ml, 266 mmol) was added dropwise. The mixture was stirred at room temperature for 16 h, the volatile components evaporated in-vacuo and the residue suspended in dichloromethane. The resulting mixture was added slowly (over 3 minutes) to a cooled (0° C.) mixture of ammonium hydroxide (33% solution in water, 750 ml) and potassium carbonate (56.5 g, 410 mmol). The resulting biphasic suspension was stirred for 1 h. The dichloromethane was then removed in-vacuo and the pH adjusted to 8-9 with aqueous hydrochloric acid. The suspension was then filtered off, washing with water (2×300 ml), heptane (2×300 ml) and diethyl ether (2×300 ml) and dried to afford the title compound as a sandy coloured solid (66.5 g, 195 mmol).

### Step I: 7-Chloro-1-[(1.1-dioxo-hexahydrothiopyran-4-yl)methyl]-3-([1,3,4]-oxathiazol-2-on-5-yl)-1H-indole

A mixture of 7-chloro-1-[(1,1-dioxo-hexahydrothiopyran-4-yl)methyl]-1H-indole-3-carboxamide (10.0 g, 29.3 mmol) and chlorocarbonylsulfenylchloride (5.05 ml, 60.9 mmol) in tetrahydrofuran (150 ml) was refluxed gently under nitrogen with stirring for 3 h. The reaction mixture was concentrated in vacuo, cooled and the solid filtered off. The solid was taken up in acetone and the mixture was concentrated in vacuo, cooled and the resulting buff coloured solid filtered off and dried to afford the title compound (8.7 g, 21.8 mmol).

### Step J: 7-Chloro-1-[(1,1-dioxo-hexahydrothiopyran-4-yl)methyl]-3-[(5-ethylcarboxyl)-([1,2,4]thiadiazol-3-yl)]-1H-indole: approx. 1:1 mixture with 7-chloro-3-cyano-1-[(1,1-dioxo-hexahydrothiopyran-4-yl)methyl]-1H-indole

A mixture of 7-chloro-1-[(1,1-dioxo-hexahydrothiopyran-4-yl)methyl]-3-([1,3,4]-oxathiazol-2-on-5-yl)-1H-indole (8.3 g, 20.8 mmol) and ethylcyanoformate (20 ml, 202 mmol) in mixed xylenes (200 ml) was heated at vigorous reflux for 3 h. The resulting solution was concentrated in vacuo, cooled and diluted with heptane until no further precipitation occurred. The resulting solid was filtered off, washing with heptane and dried to afford the title mixture as a buff coloured solid (8.2 g)

### Step K: 7-Chloro-1-[(1.1-dioxo-hexahydrothiopyran-yl-4)methyl]-3-[(5-hydroxymethyl)-([1,2,4]thiadiazol-3-yl)]-1H-indole

To a solution of the above mixture of 7-chloro-1-[(1,1-dioxo-hexahydrothio-pyran-4-yl)methyl]-3-[(5-ethylcarboxy)-([1,2,4]thiadiazol-3-yl)]-1H-indole and 7-chloro-3-cyano-1-[(1,1-dioxo-hexahydrothiopyran-4-yl)methyl]-1H-indole (8.0 g) in dichloromethane/methanol (1:1; 240 ml) at room temperature was added sodium borohydride (1.34 g, 35.4 mmol) portionwise over 5 minutes. The reaction was stirred for 15 minutes. Acetone (20 ml) was then added and the mixture stirred for a further 5 minutes. The mixture was concentrated in vacuo to low volume and diluted with water until no further precipitation occurred. The precipitate was filtered off, washing with water and air dried. The solid was dissolved in dichloromethane (200 ml), washed with water (100 ml), brine (100 ml), dried over sodium sulfate and filtered. The solution was concentrated in vacuo. The title compound crystallised out on standing and was filtered off (4.5 g, 10.9 mmol). Further concentration of the filtrate resulted in crystallisation of the nitrile that was carried through from the previous step, 7-chloro-3-cyano-1-[(1,1-dioxo-hexahydrothiopyran-4-yl)methyl]-1H-indole (1.7 g).

### Step L: 7-Chloro-1-[(1.1-dioxo-hexahydrothiopyran-4-yl)methyl]-3-{5-[(methanesulfonyloxy)methyl]-([1,2,4]-thiadiazol-3-yl)}-1H-indole

To a suspension of 7-chloro-1-[(1,1-dioxo-hexahydrothiopyran-4-yl)methyl]-3-[(5-hydroxymethyl)-([1,2,4]thiadiazol-3-yl)]-1H-indole (4.5 g, 10.9 mmol) in dichloromethane (200 ml) was added N,N-diisopropylethylamine (3.7 ml, 21.4 mmol) followed by methanesulfonyl chloride (1.01 ml, 13.1 mmol) dropwise over 2-3 minutes. The reaction was stirred for 15 minutes, then quenched with ice cold water and stirred for a further 10 minutes. The layers were separated and the organic phase washed with water (100 ml), brine (100 ml), dried over sodium sulfate and filtered. The solvent was removed in vacuo and the residue re-crystallised from acetone to afford the title compound as a pink solid (4.2 g, 8.6 mmol).

### Step M: (S)-7-Chloro-3-[(5-{[3-N-(2-hydroxyethyl)carboxamido]piperidin-1-yl}methyl)-([1,2,4]-thiadiazol-3-yl)]-1-(1,1-dioxo-hexahydrothiopyran-4-yl)methyl-1H-indole, hydrochloride salt

A mixture of 7-chloro-1-[(1,1-dioxo-hexahydrothiopyran-4-yl)methyl]-3-{5-[(methane-sulfonyloxy)methyl]-([1,2,4]-thiadiazol-3-yl)}1H-indole (245 mg, 0.5 mmol), (S)—N-(2-hydroxyethyl)nipecotamide (103 mg, 0.6 mmol) [prepared from standard amide coupling of commercial (S)-Boc-nipecotic acid and ethanolamine] and potassium carbonate (103 mg, 0.75 mmol) in acetone (10 ml) was heated at reflux for 5 h. As the reaction was incomplete, additional (S)—N-(2-hydroxyethyl)nipecotamide (40 mg) was added and reflux continued for a further 2 h. After filtering off inorganics, solvent was removed in vacuo and the residue partitioned between dichloromethane and water. The crude product was then filtered through a 5 g Strata™ SCX giga tube. The tube was washed with methanol and then eluted with 2 M ammonia in methanol. The methanolic ammonia solution was concentrated in vacuo and the obtained residue was purified by column chromatography eluting with 4-6% (v/v) ethanol in dichloromethane to give the free base of the title compound. Addition of hydrogen chloride (1M solution in diethyl ether) to a solution of the free base in dichloromethane (5 ml) followed by precipitation twice from dichloromethane plus trace methanol with ether afforded the hydrochloride salt as a noncrystalline solid, 225 mg (0.37 mmol). EsIMS: m/z 566.5 [M+H]⁺. [α]^{D}−3.37°, 1.78 mg/mL in MeOH.

### Experiment 1

### In Vitro Determination of Efficacy and Potency at the Human CB1 Receptor Expressed in CHO Cells

Chinese Hamster Ovary (CHO) cells expressing the human CB1 receptor and a luciferase reporter gene were suspended in DMEM/F12 Nut Mix without phenol red, containing penicillin/streptomycin (50U/50 μg/ml) and fungizone (1 μg/ml). Cells were seeded into white walled, white bottomed 96 well plates at a density of 3×10⁴ cells per well (100 μl final volume) and incubated overnight (approximately 18 hrs at 37° C., 5% CO₂ in air) prior to assay. The test compound (10 mM solution in DMSO) was diluted in DMEM/F12 Nut Mix (w/o phenol red) containing 3% bovine serum albumin (BSA) to give a concentration range of 0.1 mM to 1 nM. 10 μl of each dilution was added to the relevant wells in the cell plate to give a final concentration range of 10 μM to 0.1 nM. Plates were incubated for 5 hours at 37° C. before addition of 100 μl LucLite reagent to each well (reconstituted as per manufacturer's instructions). Plates were sealed with a top seal and counted on the Packard TopCount (single photon counting, 0.01 minute count time, no count delay). Data was analysed using curve fitting and a minimum sum of squares method to produce EC₅₀ values. Maximal response (efficacy) was expressed as a percentage relative to the maximal response (100%) obtained with CP 55940.

The EC₅₀ value for compound 1a was 94 nM with an efficacy of 67%.

The EC₅₀ value for compound 2 was 23 nM with an efficacy of 42%.

### Experiment 2

### In-Vitro Determination of Efficacy and Potency at the Human CB2 Receptor Expressed in CHO Cells

Chinese Hamster Ovary (CHO) cells expressing the human CB2 receptor were suspended in HAMS F-12 containing 1 mM 3-isobutyl-1-methylxanthine (IBMX) and seeded into white walled, white bottomed 96 well plates at a density of 2×10⁴ cells per well (20 μl final volume), immediately prior to assaying. The test compound (10 mM solution in dimethylsulfoxide (DMSO)) was initially diluted 100-fold in DMSO to give a stock concentration of 0.1 mM and then further diluted in phosphate buffered saline (PBS) to provide a final concentration range of 1 μM to 0.01 nM. The compound was incubated in the presence of CB2 cells at 37° C. for 30 min, prior to a 30 min incubation with 1 μM forskolin (final concentration). cAMP measurement was performed using a DiscoveRx cAMP XS EFC assay kit, in accordance with the manufacturer's instructions. Plates were counted on the Packard TopCount (single photon counting, 0.01 minute count time, no count delay). Data was analysed using curve fitting and a minimum sum of squares method to produce EC₅₀ values. Maximal response (efficacy) was expressed as a percentage relative to the maximal response (100%) obtained with CP 55940.

The EC₅₀ value for compound 1a was 3.5 nM with an efficacy of 107%.

### Experiment 3

### Pharmacokinetics and Brain Penetration in Mice Following i.v. Administration

The ratio of brain to plasma concentrations in mice indicates the ability of a compound to cross the blood-brain barrier. The total brain and plasma concentrations following an intravenous dose of cannabinoid receptor agonist were determined as described below.

### Materials and Methods

Test compound 1a was dissolved in Milli-Q water to give 0.6 μmol/ml dosing solution. A bolus intravenous dose (5 ml/kg; 3 μmol/kg) was administered via the tail vein. Male ICR mice (Harlan, UK) in 8 groups of 4 animals were dosed as above and were terminated after 1, 5, 15, 30, 60, 120, 240 and 360 minutes.

Test compound 2 was dissolved in 2.6% glycerol_{(aq)} to give 0.6 μmol/ml dosing solution. A bolus intravenous dose (5 ml/kg; 3 μmol/kg) was administered via the tail vein.

Male ICR mice (Harlan, UK) in 3 groups of 3 animals were dosed as above and were terminated after 5, 15 and 60 minutes.

Following termination, blood was removed by cardiac puncture into EDTA-containing tubes. Plasma was harvested by centrifugation (3200×rcf, 4° C., 10 min) and stored at −20° C. until sample analysis by LC/MS/MS. Brains were rinsed 3 times in PBS and pooled brains from each time-point were stored at −20° C. until sample analysis by LC/MS/MS.

Plasma standard curves (1-1000 ng/ml) and study samples were prepared using a Tecan Genesis robot. Briefly, 50 μl of plasma (samples and standards) were added to 150 μl acetonitrile containing a known concentration of a suitable internal standard. Samples were then centrifuged (3200×rcf, 4° C.) and the supernatants analysed by LC/MS/MS.

ICR mouse brain standard curves (10-10000 ng/g for compound 1a and 1-1000 ng/g for compound 2) and samples were prepared manually. Control and pooled ICR brain samples were homogenised in 3 volumes (w/v) of ice-cold PBS. To 200 μl of brain homogenate (samples and standards) was added 600 μl acetonitrile containing a known concentration of a suitable internal standard. Samples were then centrifuged (3200×rcf, 4° C., 10 min) and the supernatants analysed by LC/MS/MS.

Plasma and brain samples were analysed using a PE Sciex 3000 mass spectrometer using a Luna C18 (30×2 mm) hpic column. Pharmacokinetic information was derived from the plasma concentrations of the test compound using WinNonLin™ software (Pharsight, USA). All data analysis is based on concentration data within the quantifiable range.

### Results

Following a 3 μmol/kg intravenous dose, the maximum measured concentrations of compound 1a were 2515 ng/ml and 43 ng/g in plasma and brain homogenate, respectively. The brain to plasma ratio of Compound 1a was 0.02, based on measured Cₘₐₓ concentrations.

Following a 3 μmol/kg intravenous dose, the maximum measured concentrations of compound 2 were 528 ng/ml and 18 ng/g in plasma and brain homogenate, respectively. The brain to plasma ratio of Compound 2 was 0.03, based on measured Cₘₐₓ concentrations.

### Experiment 4

### Tail Flick Latency in Mice Following i.v. Administration: Compound 1a

The mouse Tail Flick test is a threshold model of antinociception. In this assay mice are placed onto the tail flick apparatus and their tails exposed to a focused beam of radiant heat. The mouse reacts to the noxious thermal stimulus by flicking its tail away from the heat source. An increase in the latency to respond to this noxious stimulus can be interpreted as an antinociceptive response.

The aim of this study was to determine if the net antinociception resulting from the co-administration of an opioid receptor agonist with the peripherally restricted CB1 receptor agonist is greater than that obtainable with the opioid receptor agonist alone. In this study a dose of Compound 1a that had no effect when administered alone in the Tail Flick test was combined with a dose of opioid receptor agonist producing about a 50% effect, to determine if the effect of the opioid could be potentiated.

### Materials and Methods

Male ICR mice weighing 22-32 g were weighed and randomly assigned to a treatment group. Mice were previously trained to sit still in a tail flick apparatus (Ugo Basile, Italy) whilst tail flick latency was measured. The tail was exposed to a focused beam of radiant heat at a point approximately 2.5 cm from the tip. Tail flick latency was defined as the interval between the appliance of the thermal stimulus and withdrawal of the tail. A 12 second cut-off was employed to prevent tissue damage.

### Experiment 4a

### The Effect of the CB1 Receptor Agonist Compound 1a Alone

Four groups of eight mice were treated with vehicle or one of three doses of Compound 1a administered intravenously (vehicle: 10% Tween 80 in saline 9 g/l; injection volume 10 ml/kg). Tail flick latency was measured before i.v. administration of vehicle or the test compound (3.0, 10.0 and 30.0 μmol.kg⁻¹) and at 20, 40 and 60 min after compound administration.

### Experiment 4b

### The Effect of the μ Opioid Receptor Agonist Morphine in Combination with Compound 1a

Morphine (1.56 μmol.kg⁻¹) at a dose that produced approximately a 50% increase in tail flick latency compared to the maximum possible effect (MPE) was combined with doses of Compound 1a (0.1, 0.3 and 1.0 μmol.kg⁻¹) that were not antinociceptive alone in the tail flick test. Compounds were prepared at twice the final concentration required for testing. Equal volumes of compounds were mixed and given as a final volume of 10 ml.kg⁻¹.

### Experiment 4c

### The Effect of the μ Opioid Receptor Agonist Fentanyl in Combination with Compound 1a

Fentanyl (0.05 μmol.kg⁻¹) at a dose that produced approximately a 50% increase in tail flick latency compared to the MPE was combined with doses of Compound 1a (0.1, 0.3 and 1.0 μmol.kg⁻¹) that were not antinociceptive alone in the tail flick test. Compounds were prepared at twice the final concentration required for testing. Equal volumes of compounds were mixed and given as a final volume of 10 ml.kg⁻¹.

### Experiment 4d

### The Effect of the μ Opioid Receptor Agonist Codeine in Combination with Compound 1a

Codeine (25.5 μmol.kg⁻¹) at a dose that produced approximately a 50% increase in tail flick latency compared to the MPE was combined with doses of Compound 1a (0.1, 0.3 and 1.0 μmol.kg⁻¹) that were not antinociceptive alone in the tail flick test. Compounds were prepared at twice the final concentration required for testing. Equal volumes of compounds were mixed and given as a final volume of 10 ml.kg⁻¹.

An additional group of mice was treated with Compound 1a (1.0 μmol.kg⁻¹) to confirm that this dose had no effect when given alone.

Doses of compounds tested, group numbers and mean tail flick latency calculated+s.e.m. at Tₘₐₓ are shown in Table 1.

### Data Analysis

Data were plotted as mean±s.e.m. The time of maximum effect for each mouse in the two top dose groups was determined and these values averaged to calculate the mean time of maximum effect. For analytical purposes Tₘₐₓ was defined as the time point closest to this averaged value. Tₘₐₓ data were used for statistical comparisons.

For the dose response experiment, Tₘₐₓ data were compared between groups using the Kruskal-Wallis one-way analysis of variance, a non-parametric statistical test. If statistical significance (P<0.05) was observed the vehicle group and each of the treatment groups was compared using a non-parametric post-hoc test, the Dunn's test (Unistat 5.0 software).

For the interaction experiments, Tₘₐₓ data for each of the compound treated groups were compared using the Kruskal-Wallis one-way analysis of variance. If statistical significance (P<0.05) was observed, the opioid plus vehicle group and each of the combination treatment groups was compared using a non-parametric post-hoc test, the Dunn's test (Unistat 5.0,software).

To determine if a single dose of Compound 1a at 1.0 μmol.kg⁻¹ had an effect on tail flick latency the Kruskal-Wallis one-way analysis of variance was carried out where the factor was time.

Tail flick latency at the Tₘₐₓ were expressed as the % maximum possible effect (% MPE) where

$$\% M P E = \frac{Post \text{-} drug latency \text{-} baseline latency \times 100}{Cut \text{-} off latency ( 12 s ) - baseline latency}$$

Morphine, Fentanyl and Codeine were all purchased from Sigma Aldrich UK and were dissolved in saline. Compound 1a was dissolved in 10% Tween 80 in saline.

### Results (Table 1)

Compound 1a administered i.v. at a dose of 3 μmol.kg⁻¹ had no effect on tail flick latency. Higher doses of Compound 1a increased the tail flick latency in a dose-dependent manner, with the maximum effect occurring at 40 min post-injection. After administration of 30 μmol.kg⁻¹ of Compound 1a the tail flick latency was 7.15±0.88 s, this compared to a tail flick latency of 3.31 s+0.22 s after vehicle treatment. This effect of Compound 1a was significantly different (Dunn's test, P<0.05) from vehicle treatment. In the dose response experiment a dose of 3 μmol.kg⁻¹ had no effect on tail flick latency. In addition, when tested, a 1.0 μmol.kg⁻¹ dose of Compound 1a did not increase the tail flick latency. Doses of 0.1, 0.3 & 1.0 μmol.kg⁻¹ were selected for the combination experiments.

Compound 1a at 0.1, 0.3 and 1.0 μmol.kg⁻¹ co-administered i.v. with morphine (1.56 μmol.kg⁻¹), fentanyl (0.05 μmol.kg⁻¹) or codeine (25.5 μmol.kg⁻¹) increased tail flick latencies in a dose-dependent manner, with Tₘₐₓ occurring at 40 min post-injection for morphine and at 20 min post-injection for fentanyl and codeine, respectively.

The effect of the combination of the top dose of Compound 1a (1.0 μmol.kg⁻¹) and morphine, fentanyl or codeine was significantly different from the tail flick latency recorded after administration of the opioid on its own.

### Conclusions

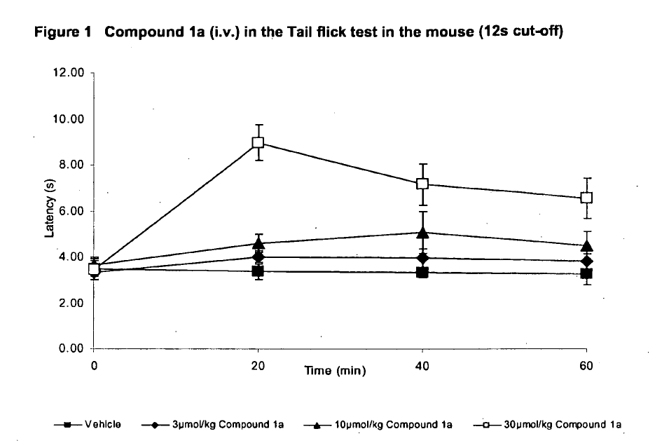

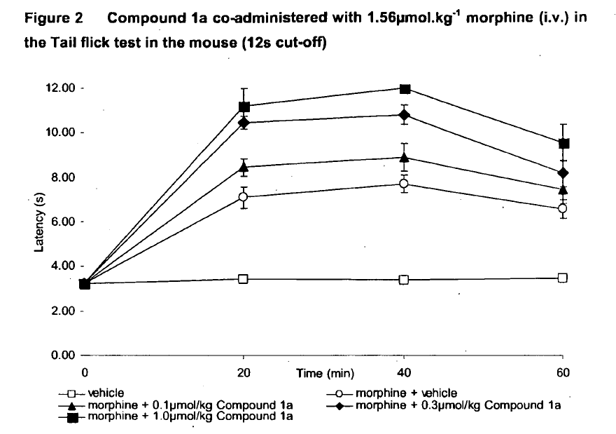

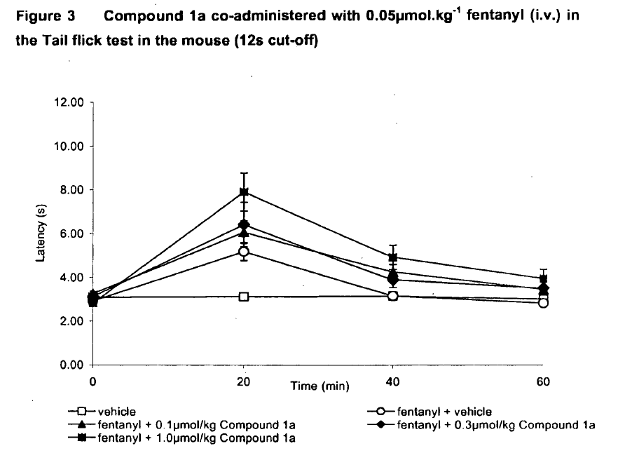

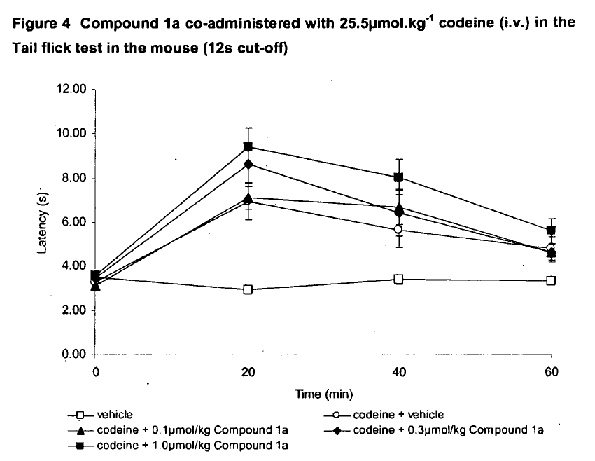

Compound 1a administered at doses below 3.0 μmol.kg⁻¹ had no effect on tail flick latency (see FIG. 1). Each of the opioid receptor agonists increased tail flick latency. A dose-dependent potentiation of this effect was observed when Compound 1a was administered at doses of 0.1, 0.3 and 1.0 μmol.kg⁻¹ in combination with each of the opioid receptor agonists morphine (FIG. 2), fentanyl (FIG. 3) and codeine (FIG. 4).

TABLE 1

| | | | | | Tail flick | |
|---|---|---|---|---|---|---|
| | | | | Number | latency (s) + | |
| Opioid | Dose | Cannabinoid | Dose | of | s.e.m. at | % of |
| or vehicle | (μmol · kg⁻¹) | or vehicle | (μmol · kg⁻¹) | animals | Tmax | MPE |
| — | — | 10% tween 80 | 10 ml · kg − 1 | 8 | 3.31 ± 0.22 | — |
| — | — | Compound 1a | 3.0 | 8 | 3.95 ± 0.40 | 7.4 |
| — | — | Compound 1a | 10.0 | 8 | 5.08 ± 0.90 | 20.4 |
| — | — | Compound 1a | 30.0 | 8 | 7.15 ± 0.88 * | 44.2 |
| Saline | 5 ml · kg − 1 | 10% tween 80 | 5 ml · kg − 1 | 8 | 3.88 ± 0.88 | — |
| morphine | 1.56 | 10% tween 80 | 5 ml · kg − 1 | 8 | 7.75 ± 0.81 | 47.7 |
| morphine | 1.56 | Compound 1a | 0.1 | 8 | 9.04 ± 1.00 | 63.5 |
| morphine | 1.56 | Compound 1a | 0.3 | 8 | 9.71 ± 0.70 | 71.8 |
| morphine | 1.56 | Compound 1a | 1.0 | 8 | 11.94 ± 0.06⁺ | 99.3 |
| Saline | 5 ml · kg − 1 | 10% tween 80 | 5 ml · kg − 1 | 8 | 3.11 ± 0.16 | — |
| Fentanyl | 0.05 | 10% tween 80 | 5 ml · kg − 1 | 8 | 5.18 ± 0.41 | 23.2 |
| Fentanyl | 0.05 | Compound 1a | 0.1 | 8 | 6.06 ± 0.52 | 33.2 |
| Fentanyl | 0.05 | Compound 1a | 0.3 | 8 | 6.41 ± 1.01 | 37.1 |
| Fentanyl | 0.05 | Compound 1a | 1.0 | 8 | 7.90 ± 0.86 ⁺ | 53.9 |
| Saline | 5 ml · kg − 1 | 10% tween 80 | 5 ml · kg − 1 | 8 | 2.95 ± 0.16 | — |
| Saline | 5 ml · kg − 1 | Compound 1a | 1.0 | 8 | 3.63 ± 0.22 | 7.5 |
| Codeine | 25.5 | 10% tween 80 | 5 ml · kg − 1 | 8 | 6.95 ± 0.84 | 44.2 |
| Codeine | 25.5 | Compound 1a | 0.1 | 8 | 7.11 ± 0.52 | 46.0 |
| Codeine | 25.5 | Compound 1a | 0.3 | 8 | 8.64 ± 0.82 | 62.9 |
| Codeine | 25.5 | Compound 1a | 1.0 | 8 | 9.41 ± 0.85 ⁺ | 71.4 |

* denotes that the effect was significantly greater than in vehicle-treated mice (P < 0.05, Dunn's post-test).
⁺ denotes that the effect was significantly greater than in opiod receptor agonist only treated mice: morphine, fentanyl and codeine (P < 0.05, Dunn's post-test).

### Experiment 5

### Reversal of Mouse Tail Flick Latency Potentiation by a Selective CB1 Receptor Antagonist

The aim of this study was to determine if the potentiation of antinociception resulting from the co-administration of the peripherally restricted CB1 receptor agonist, Compound 1a, with morphine could be reversed by pre-treatment with the selective CB1 receptor antagonist SR141716A (Barth, F et al., Eur. Patent Application EP-00656354, 1995; Rinaldi-Carmona M. et al., FEBS Lett. 350: 240-244, 1994).

Five groups of eight mice were pre-treated with vehicle (5% mulgofen in saline) or SR141716A (3.0 μmol.kg⁻¹; injection volume 10 ml.kg⁻¹) administered subcutaneously (s.c.) 20 min prior to intravenous administration of the test combination. For i.v. dosing, equal volumes of compounds were mixed and given as a final volume of 10 ml.kg⁻¹: either saline (5 ml.kg⁻¹) or morphine (1.51 μmol.kg⁻¹ in saline; 5 ml.kg⁻¹), combined with either 10% Tween 80 in saline (5 ml.kg⁻¹) or Compound 1a (1.0 μmol.kg⁻¹ in 10% Tween 80 in saline; 5 ml.kg⁻¹). Tail flick latency was measured before compound administration and at 20, 40, 60 and 90 min after i.v. administration of compounds.

Doses of compounds tested, group numbers and mean tail flick latency calculated+s.e.m. at Tₘₐₓ are shown in Table 2.

Data analysis was carried out as described for Experiment 4.

### Results (Table 2)

Morphine after i.v. administration increased the tail flick latency from 3.53±0.13 to 6.78±0.27 s in mice that were pre-treated with vehicle (s.c.). Compound 1a at 1.0 μmol.kg⁻¹ and morphine at 1.51 μmol.kg⁻¹ in combination increased the tail flick latency to 11.33±0.36 s. This effect was significantly different to that observed after administration of morphine in combination with vehicle (Dunn's test P<0.01). This potentiation was blocked by pre-treatment with the selective CB1 receptor antagonist SR141716A (s.c.); in animals that received SR141716A followed by morphine in combination with Compound 1a, the tail flick latency was 6.75±0.77 s. SR141716A alone had no effect on the tail flick latency.

### Conclusions

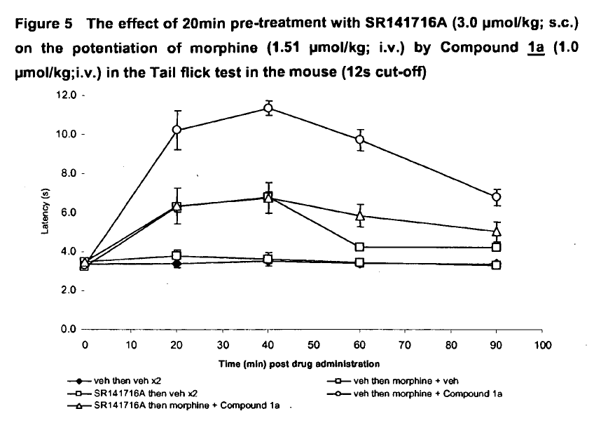

The potentiation of the effect of morphine in the mouse Tail Flick test by Compound 1a was fully reversed by pre-treatment with the selective CB1 receptor antagonist, SR141716A (FIG. 5). This result indicates that the observed potentiation is the result of a pharmacodynamic, rather than a pharmacokinetic interaction and that the effect is mediated by the CB1 receptor.

TABLE 2

| CB1 | | | | | | | | |
|---|---|---|---|---|---|---|---|---|
| antagonist | | opioid | | cannabinoid | | | Tail flick | % of |
| or vehicle | | or vehicle | | or vehicle | | | latency (s) + | MPE |
| s.c. | Dose | i.v. | Dose | i.v. | Dose | | s.e.m. at | at |
| (−20 min) | (μmol · kg⁻¹) | (0 min) | (μmol · kg⁻¹) | (0 min) | (μmol · kg⁻¹) | n | Tmax | Tmax |
| 5% mulgofen | 10 ml · kg⁻¹ | saline | 5 ml · kg⁻¹ | 10% tween 80 | 5 ml · kg⁻¹ | 8 | 3.53 ± 0.13** | — |
| SR141716A | 3.0 | saline | 5 ml · kg⁻¹ | 10% tween 80 | 5 ml · kg⁻¹ | 8 | 3.61 ± 0.35** | 0.9 |
| 5% mulgofen | 10 ml · kg⁻¹ | morphine | 1.51 | 10% tween 80 | 5 ml · kg⁻¹ | 8 | 6.78 ± 0.27 | 38.3 |
| 5% mulgofen | 10 ml · kg⁻¹ | morphine | 1.51 | Compound 1a | 1.0 | 8 | 11.33 ± 0.36** | 92.1 |
| SR141716A | 3.0 | morphine | 1.51 | Compound 1a | 1.0 | 8 | 6.75 ± 0.77 | 38.0 |

**denotes that the effect was significantly different from morphine and vehicle only treated mice (P < 0.01, Dunn's post-test).

### Experiment 6

### Tail Flick Latency in Mice Following i.v. Administration: Compound 2

The aim of this study was to determine if the net antinociception resulting from the co-administration of an opioid receptor agonist with the peripherally restricted CB1 receptor agonist Compound 2, is greater than that obtainable with the opioid receptor agonist alone. In this study a dose of Compound 2 that had no effect when administered alone in the Tail Flick test was combined with a dose of opioid receptor agonist producing about a 50% effect, to determine if the effect of the opioid could be potentiated.

### Materials and Methods

As for Experiment 4.

### Experiment 6a

### The Effect of the CB1 Receptor Agonist Compound 2 Alone

Four groups of eight mice were treated with vehicle or one of three doses of Compound 2, administered intravenously (vehicle: 10% Tween 80 in saline 9 g/l; injection volume 10 ml/kg). Tail flick latency was measured before i.v. administration of vehicle or the test compound (10.0, 30.0 and 60.0 μmol.kg⁻¹) and at 20, 40 and 60 min after compound administration.

### Experiment 6b

### The Effect of the μ Opioid Receptor Agonist Morphine in Combination with Compound 2

Morphine (1.56 μmol.kg⁻¹) at a dose that produced approximately a 50% increase in tail flick latency compared to the maximum possible effect (MPE) was combined with doses of Compound 2 (3.0, 10.0 and 30.0 μmol.kg⁻¹) that were not antinociceptive alone in the tail flick test. Compounds were prepared at twice the final concentration required for testing. Equal volumes of compounds were mixed and given as a final volume of 10 ml.kg⁻¹. An additional group of mice was treated with Compound 2 (30 μmol.kg⁻¹) to confirm that this dose had no effect when given alone.

Doses of compounds tested, group numbers and mean tail flick latency calculated+s.e.m. at Tₘₐₓ are shown in Table 3.

### Data Analysis

### As for Experiment 4.

### Results (Table 3)

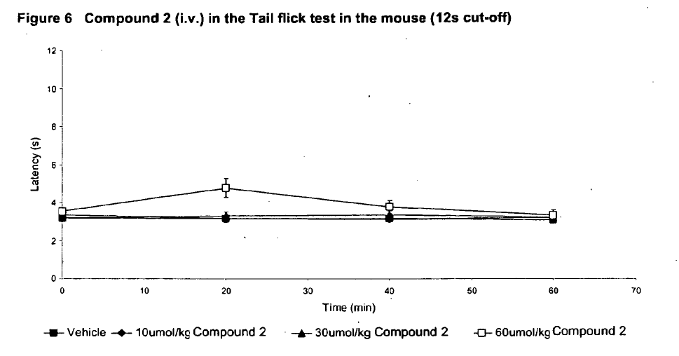

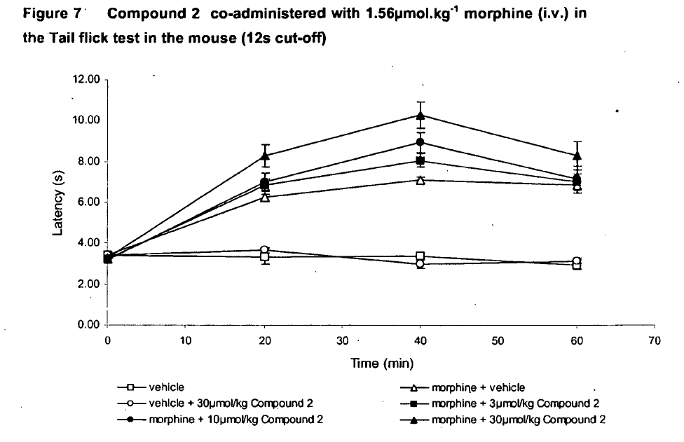

Compound 2 administered i.v. at a dose of 30 μmol.kg⁻¹ had no effect on tail flick latency (FIG. 6). A dose of 60 μmol.kg⁻¹ significantly increased the tail flick latency (Dunn's test, P<0.05), with the maximum effect occurring at 20 min post-injection. Compound 2 at 3.0, 10.0 & 30.0 μmol.kg⁻¹ co-administered i.v. with morphine (1.56 μmol.kg⁻¹) increased tail flick latency in a dose-dependent manner, with Tₘₐₓ occurring at 40 min post-injection (FIG. 7).

The effect of the combination of the top dose of Compound 2 (30.0 μmol.kg⁻¹) and morphine was significantly different from the tail flick latency recorded after administration of the opioid on its own. In addition, a 30.0 μmol.kg⁻¹ dose of Compound 2 was included in the experiment and did not increase the tail flick latency.

### Conclusions

Compound 2 administered at a dose of 30.0 μmol.kg⁻¹ had no effect on tail flick latency. A dose-dependent potentiation of the effect of morphine on tail flick latency was observed when Compound 2 was administered at doses of 3.0, 10.0 and 30.0 μmol.kg⁻¹ in combination with morphine.

TABLE 3

| | | | | | Tail flick | |
|---|---|---|---|---|---|---|
| | | | | Number | latency (s) + | |
| Opioid | Dose | Cannabinoid | Dose | of | s.e.m. at | % of |
| or vehicle | (μmoL · kg⁻¹) | or vehicle | (μmol · kg⁻¹) | animals | Tmax | MPE |
| — | — | 10% tween 80 | 10 ml · kg⁻¹ | 8 | 3.18 ± 0.15 | — |
| — | — | Compound 2 | 10.0 | 8 | 3.18 ± 0.20 | −2.23 |
| — | — | Compound 2 | 30.0 | 8 | 3.30 ± 0.23 | 1.12 |
| — | — | Compound 2 | 60.0 | 8 | 4.78 ± 0.49* | 13.87 |
| — | — | Saline | 10 ml · kg⁻¹ | 8 | 3.39 ± 0.14 | — |
| Saline | 5 ml · kg⁻¹ | Compound 2 | 30.0 | 8 | 2.98 ± 0.19 | −5.0 |
| morphine | 1.56 | Saline | 5 ml · kg¹⁻ | 8 | 7.09 ± 0.17 | 42.9 |
| morphine | 1.56 | Compound 2 | 3.0 | 8 | 8.05 ± 0.34 | 54.9 |
| morphine | 1.56 | Compound 2 | 10.0 | 8 | 8.93 ± 0.50 | 65.1 |
| morphine | 1.56 | Compound 2 | 30.0 | 8 | 10.25 ± 0.65⁺ | 80.0 |

*denotes that the effect was significantly greater than in vehicle-treated mice (P < 0.05, Dunn's post-test).
⁺denotes that the effect was significantly greater than in morphine only treated mice (P < 0.05, Dunn's post-test).

1. A pharmaceutical dosage form comprising an analgesic combination for simultaneous or sequential use which comprises a cannabinoid CB I receptor agonist and an opioid receptor agonist, characterized in that the cannabinoid receptor agonist is peripherally restricted having a brain Cₘₐₓ to plasma Cₘₐₓ ratio of less than 0.1.

2. The pharmaceutical dosage form of claim 1, wherein the peripherally restricted cannabinoid receptor agonist has a brain Cₘₐₓ to plasma Cₘₐₓ ratio of less than 0.05.

3. The pharmaceutical dosage form of claim 1, wherein the opioid receptor agonist is selected from alfentanil, allylprodine, alphaprodine, anileridine, benzylmorphine, bezitramide, buprenorphine, butorphanol, clonitazene, codeine, cyclorphan, desomorphine, dextromoramide, dezocine, diamorphine, diampromide, dihydrocodeine, dihydromorphine, eptazocine, ethylmorphine, fentanyl, hydrocodone, hydromorphone, hydroxypethidine, levophenacylmorphan, levorphanol, lofentanil, methadone, meperidine, methylmorphine, morphine, nalbuphine, necomorphine, normethadone, normorphine, opium, oxycodone, oxycontin, oxymorphone, pentazocine, pholcodine, profadol, sufentanil and tramadol.

4. The pharmaceutical dosage form of claim 3, wherein the opioid receptor agonist is selected from morphine, codeine, fentanyl, oxymorphine, oxycodone, hydromorphine, methadone and tramadol.

5. The pharmaceutical dosage form of claim 1, wherein the opioid receptor agonist is selected from morphine, codeine, fentanyl and tramadol.

6. (canceled)

7. A method for the treatment of pain, which comprises the simultaneous or sequential administration of a peripherally restricted cannabinoid CB1 receptor agonist having a brain Cₘₐₓ to plasma Cₘₐₓ ratio of less than 0.1 and an opioid receptor agonist.
